(19) United States
(12) Patent Application Publication
(10) Pub. No.: US 2022/0267839 A1
(43) Pub. Date: Aug. 25, 2022

(54) TARGET VALIDATION, BINDING SITE IDENTIFICATION, AND PROFILING OF RNA TARGETS

(71) Applicant: THE SCRIPPS RESEARCH INSTITUTE, La Jolla, CA (US)

(72) Inventor: Matthew D. Disney, Jupiter, FL (US)

(21) Appl. No.: 17/622,005

(22) PCT Filed: Jun. 24, 2020
(86) PCT No.: PCT/US20/70175

§ 371 Date: Dec. 22, 2021

Related U.S. Application Data
(60) Provisional application No. 62/867,582, filed on Jun. 27, 2019.

(51) Int. Cl. C12Q 1/6869; C12Q 1/6806

(52) U.S. Cl. CPC C12Q 1/6869; C12Q 1/6806

(57) ABSTRACT

The invention is directed to a method of identifying the interactions of RNA such as miRNA with small molecules interacting with RNA (SMIRNAs). A candidate SMIRNA group is associated with a photoaffinity diazirene group that can form a covalent complex with an RNA target site and with an alkyne group that can be used in subsequent “click chemistry’ reactions such as a “CuAAC” reaction, a copper-catalyzed alkyne-azide cy-cloaddition to yield a stable triazole ring. By this means, the RNA binding site of the small molecule can be identified via isolation of the RNA-targetSMIRNA covalent complex and reverse transcription of the RNA followed by DNA sequencing of the reverse transcription product. Sites on the RNA blocked during reverse transcription by the covalently bound SMIRNA are identified as terminations in the sequence compared to the native RNA.

### CLAIM OF PRIORITY

The present application claims the benefit of priority to U.S. Provisional Application No. 62/867,582 filed on Jun. 27, 2019, which application is incorporated by reference as if fully set forth herein.

### STATEMENT OF GOVERNMENT SUPPORT

This invention was made with government support under grant number GM97455 awarded by the National Institutes of Health. The government has certain rights in the invention.

### BACKGROUND

In the post genomic era, it is now realized that only 1-2% of the genorne encodes for proteins, and only a fraction of the proteorne is known as druggable. Functional studies have shown that the non-coding RNAs exhibit regulatory functions critical to cellular function.

### SUMMARY

The invention, in various embodiments, provides a method of validating the targets of small molecules in vitro and in cells and of profiling all RNA targets that bind a small molecule in vitro and in cells. RNA secondary structural motifs, such as stern loop, internal loop, bulge, pseudoknot, and G-quaduplex, are the preferred binding sites of small molecules. Targeting these motifs and affecting the function of RNAs has the potential to tackle many diseases for which the RNAs either have a direct impact on disease phenotype, or indirectly influence the translation of disease-causing proteins.

The invention provides, in various embodiments, a method of determining the binding site on an RNA molecule to which a small molecule interacting with RNA (SMIRNA) associates, comprising

forming a derivative of the small molecule interacting with RNA (SMIRNA) comprising the small molecule covalently coupled with a moiety comprising a diazirine group and a moiety comprising an alkyne group; then,

contacting the RNA with the SMIRNA derivative and exposing to UV light to activate the diazirene and bring about covalent coupling of the SMIRNA derivative to the RNA forming a SMIRNA-RNA covalent complex; then,

contacting the SMIRNA-RNA covalent complex with an azide-bearing group further comprising a pull-down group comprising a biotin moiety or beads; then,

contacting the SMIRNA-RNA covalently bonded to the biotin moiety, and a complementary pull-down entity comprising streptavidin immobilized on a bead to isolate the SMIRNA-RNA covalent complex; then,

cleaving the biotin group or the bead from the SMIRNA-RNA covalent complex to free the complex from the bead; then,

subjecting the RNA thus freed to reverse transcription and sequencing of the DNA from the reverse transcription process, to indicate the SMIRNA binding site on the RNA through the presence of a reverse transcription stop.

### BRIEF DESCRIPTION OF THE FIGURES

FIG. 1 illustrates examples of how small molecules interacting with RNA (SMIRNAs) can interact with microRNAs (miRNAs) in the cell. The term SMIR in the figure refers to a SMIRNA.

FIG. 2 shows the concept of sequence-based design of small molecules to target RNAs. The Inform process matches disease-causing RNA secondary structures with small molecules from a database of RNA secondary structures and small molecules that interact with those motifs.

FIG. 3A, 3B, 3C: FIG. 3A illustrates the binding of a RNA-binding small molecule (SMIRNA, small molecule interacting with RNA), also bearing a diazirene group and an alkyne group, to a miRNA secondary structure motif target site, and UV light-induced covalent bond formation between the diazirine group and the miRNA molecule; FIG. 3B illustrates the CuAAC click chemistry cycloaddition between the alkyne group of the small molecule and an azide group of a molecule further comprising a “pull-down” group such as a biotin moiety, shown as a large shaded circle. The site of diazirene-derived carbene coupling to the target miRNA is shown as a star. FIG. 3C illustrates the “pull-down” and isolation of the target miRNA covalently coupled with the SMIRNA.

FIG. 4 illustrates that diazirine photo crosslinking of RNA generates RT stop during reverse transcription.

### DETAILED DESCRIPTION

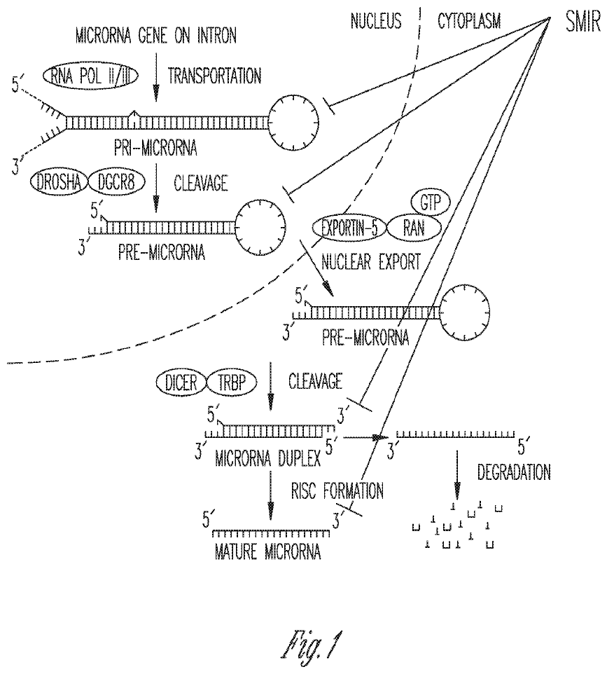

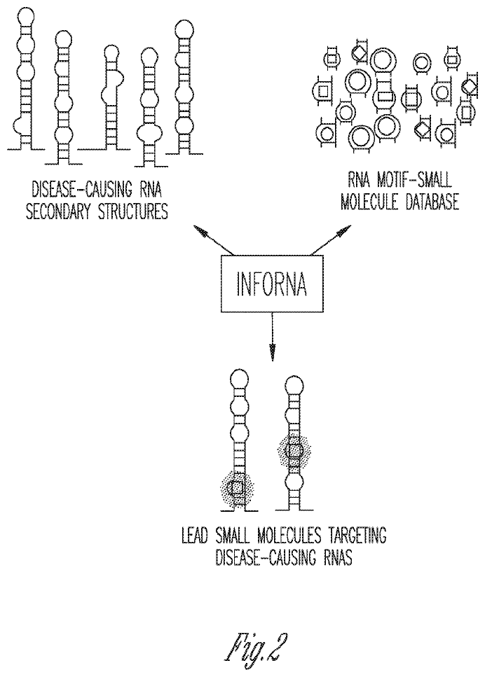

MiRNAs provide possible targets for small molecules due to their involvement in various cellular processes. Targeting miRNAs via interactions of the miRNA with small molecules interacting with RNA (SMIRNAs) offers opportunities for inhibiting the functioning of the miRNA in various ways, For example, rniRNA functions in RNA silencing and post-transcriptional regulation of gene expression, MiRNAs function via base-pairing with complementary sequences within rnRNA molecules. More specifically, targeting the Dicer and Drosha processing sites using small molecules can affect the biogenesis of the miRNA. FIG. 1 shows examples of cellular processes, some nuclear (processing of pri-miRNA and pre-miRNA) and some cytoplasmic (at the stage of mature miRNA). Because miRNAs can be involved with mRNA target cleavage, translational repression, and mRNA de-adenylation, the interaction of small molecules with miRNA can serve to have an effect on these processes.

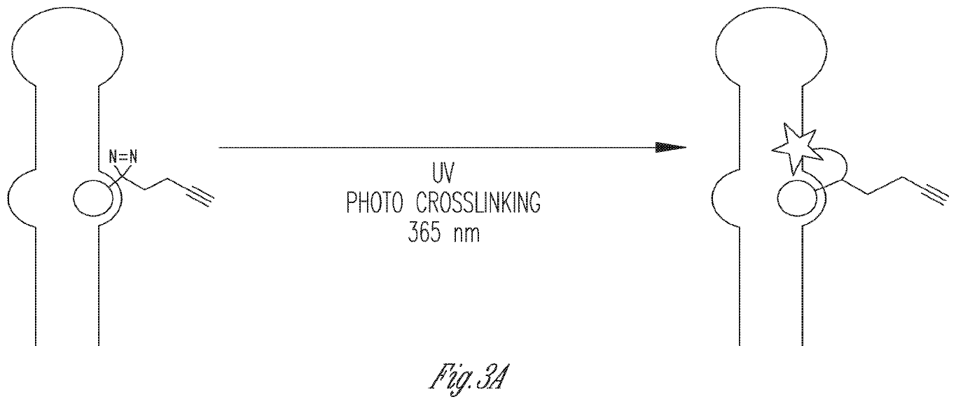

As shown in FIG. 2, the Inthrna process (See Nature Chemical Biology 2014, 10,291-291) can be used to associate small molecules that interact with one of the several types of miRNA substructures mentioned above. For example, it is known that the small molecule of structure (SMIRNA-1)

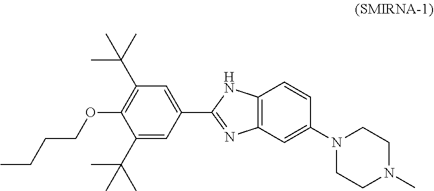 [SMILES: CCCCOc1c(C(C)(C)C)cc(-c2nc3cc(N4CCN(C)CC4)ccc3[nH]2)cc1C(C)(C)C]

interacts specifically with the rniRNA second structure motif of formula

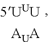

which is known to be a motif in miR-96, associated with breast, pancreatic, and other cancers. For another example, the small molecule of structure (SMIRNA-2)

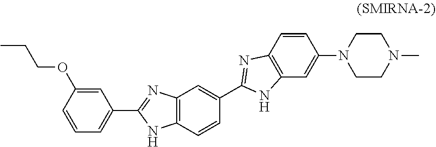 [SMILES: CCCOc1cccc(-c2nc3cc(-c4nc5ccc(N6CCN(C)CC6)cc5[nH]4)ccc3[nH]2)c1]

interacts specifically with the miRNA secondary structure motif of formula

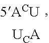

which is known to be a motif in miR-210, associated with metastatic cancers.

To identify the miRNA secondary structure targets of other RNA-binding SMIRNAs, the inventors herein provide a method, termed “Photo-Chem-CLIP-Map-Seq”, in which a small molecule that is a candidate SMIRNA group is associated with a photoaffinity diazirene group that can form a covalent complex with an RNA target site and with an alkyne group that can be used in subsequent “click chemistry’ reactions such as a “CuAAC” reaction, a copper-catalyzed alkyne-azide cycloaddition to yield a stable triazole ring. By this means, the RNA binding site of the small molecule can be identified via isolation of the RNA-target:SMIRNA covalent complex and reverse transcription of the RNA followed by DNA sequencing of the reverse transcription product. Sites on the RNA blocked during reverse transcription by the covalently bound SMIRNA are identified as terminations in the sequence compared to the native miRNA.

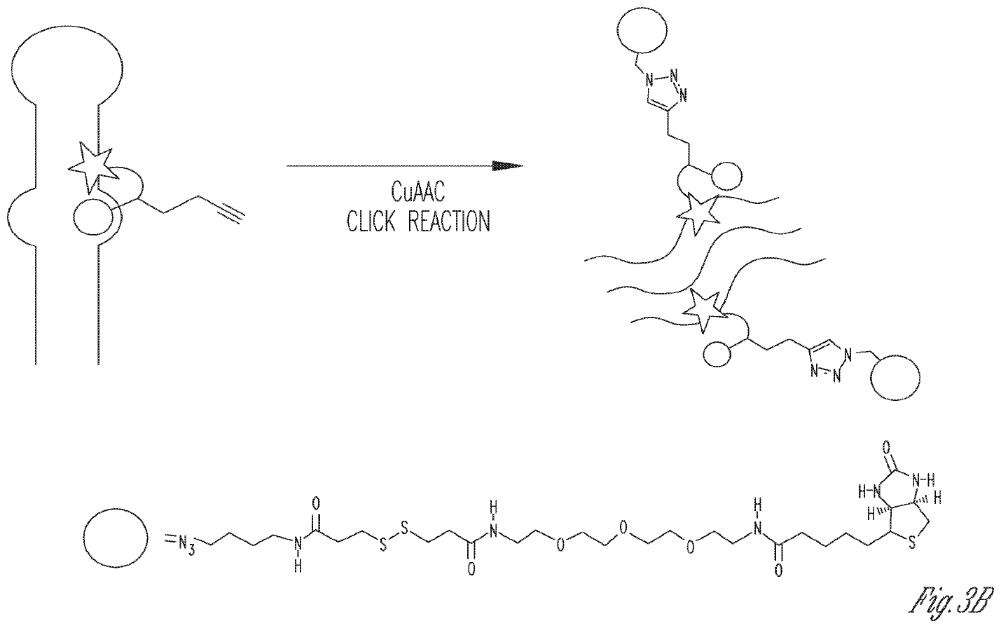

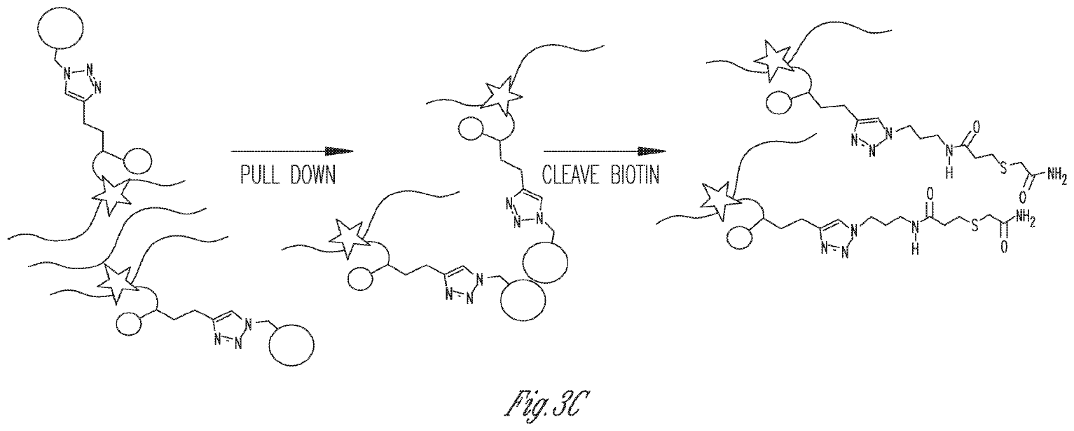

FIG. 3A illustrates the initial step in the method of covalently labeling the miRNA secondary structure target, shown as the RNA hairpin loop with bulges, binding the small molecule indicated by the solid circle, wherein the small molecule having the RNA binding properties also comprises the photoaffinity diazirene group that can react to form a covalent complex with the RNA target site and with an alkyne group that can be used in the subsequent CuAAC click chemistry reaction, A CuAAC reaction is a copper-catalyzed alkyne-azide cycloaddition reaction forming a triazole ring. The small molecule, bound to the RNA secondary structure motif, is reacted to form a covalent bond with the RNA target with which the small molecule is associated, by photoactivation of the diazirine group, e.g., with 365 nm UV light. The diazirine moiety upon photoactivation by the UV light is converted into a highly reactive electrophilic carbene, which can insert into a chemical bond of the RNA target physically proximate to the bound small molecule. The site of covalent bonding of the carbene to the miRNA target is indicated as a star.

In FIG. 3B, the second step of the process is illustrated, wherein the alkyne group from the small molecule SMIRNA undergoes a CuAAC click chemistry cycloaddition reaction with an azide group disposed on a molecule also comprising a “pull-down” group, shown as a large shaded circle in FIG. 3B. A pull-down group can be a biotin group, which can be “pulled down” by association with an immobilized streptavidin moiety disposed on a bead. The linker between the biotin group and the streptavidin comprises a disulfide linkage that can be cleaved reductively.

Alternatively, the CuAAC reaction can be carried out between the alkyne group of the SMIRNA-RNA covalent complex and an azide group disposed on a bead by a linker that comprises a disulfide bond. In this case the product of the CuAAC reaction between the SMIRNA-RNA covalent complex and an azide group disposed on a bead can be carried out directly, as no biotin-streptavidin association reaction is needed to produce a bead-bound labeled RNA.

Once the small molecule with covalently bound miRNA has been pulled down, e.g., by association of a biotin group and a streptavidin moiety, or by direct CuAAC reaction with an azide-derivatized bead., the labeled RNA can be freed of the pull-down group if necessary, and then subjected to reverse transcription and PCR DNA sequencing to identify the sites in which the reverse transcription was blocked due to the presence of the covalently bound SMIRNA.

FIG. 3C illustrates the subsequent steps in pulling down the labeled SMIRNA-RNA covalent complex, then releasing the labeled RNA by reductive cleavage of the disulfide bond in the linker connecting the biotin (bound to streptavidin on the bead) and the SMIRNA-RNA covalent complex.

The SMIRNA-RNA covalent complex is then subjected to reverse transcription to provide a cDNA complementary to the RNA, which is then sequenced. The binding site of the SMIRNA to the RNA is indicated by a transcription stop in the cDNA.

Synthetic Schemes 1 and 2, below, illustrates the preparation of exemplary SMIRNAs with diazirine and alkyne group. A candidate SMIRNA, in this case a compound of formula (SMIRNA-1), comprising an N-methylpiperazinyl-benzimidazolyl-(di-t-butyl)phenoxyl group, bears a carboxylic acid group at the terminus of the n-butoxy group to serve as a linker with the moiety bearing the alkyne group, which itself bears an amino group as a linker with the diazirine acid bearing the diazirene group. This carboxylic acid derivative of SMIRNA-1 undergoes amide formation with a moiety bearing an alkyne group provided by an N-propargyl amide of the 2,4-diaminobutyrate. Then, this product is coupled with a carboxylic acid bearing a diazirene group, to provide the SMIRNA reagent TGP-96-mono-diaz (Synthetic Scheme 1).

Alternatively, Synthetic Scheme 2 shows a compound bearing two SMIRNA-1 group coupled in an analogous manner with the moiety bearing the alkyne group and the moiety bearing the diazirine group.

Further details are provided below in the Examples section.

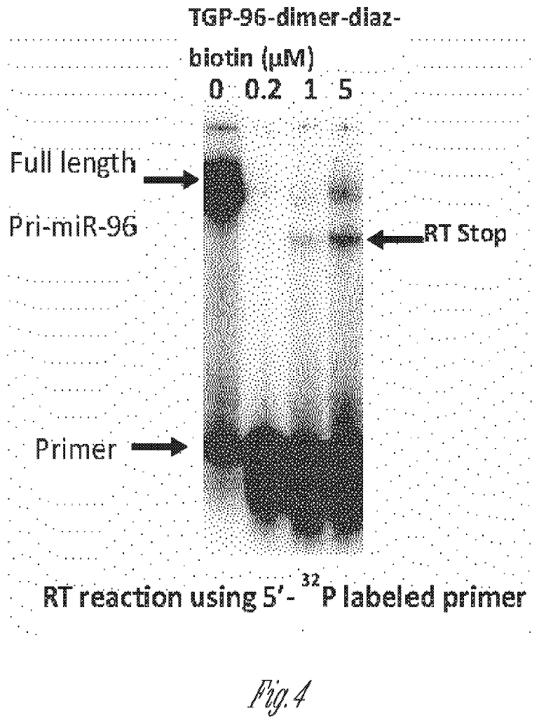

FIG. 4 illustrates how the presence of a diazirine photo crosslinking of an RNA target generates a reverse transcription (RT) stop. When the reverse transcribed cDNA product is sequenced, the deletion(s) compared to the known sequence of the RNA and its reverse transcribed cDNA indicates the binding site of the SMIRNA under evaluation.

### EXAMPLES

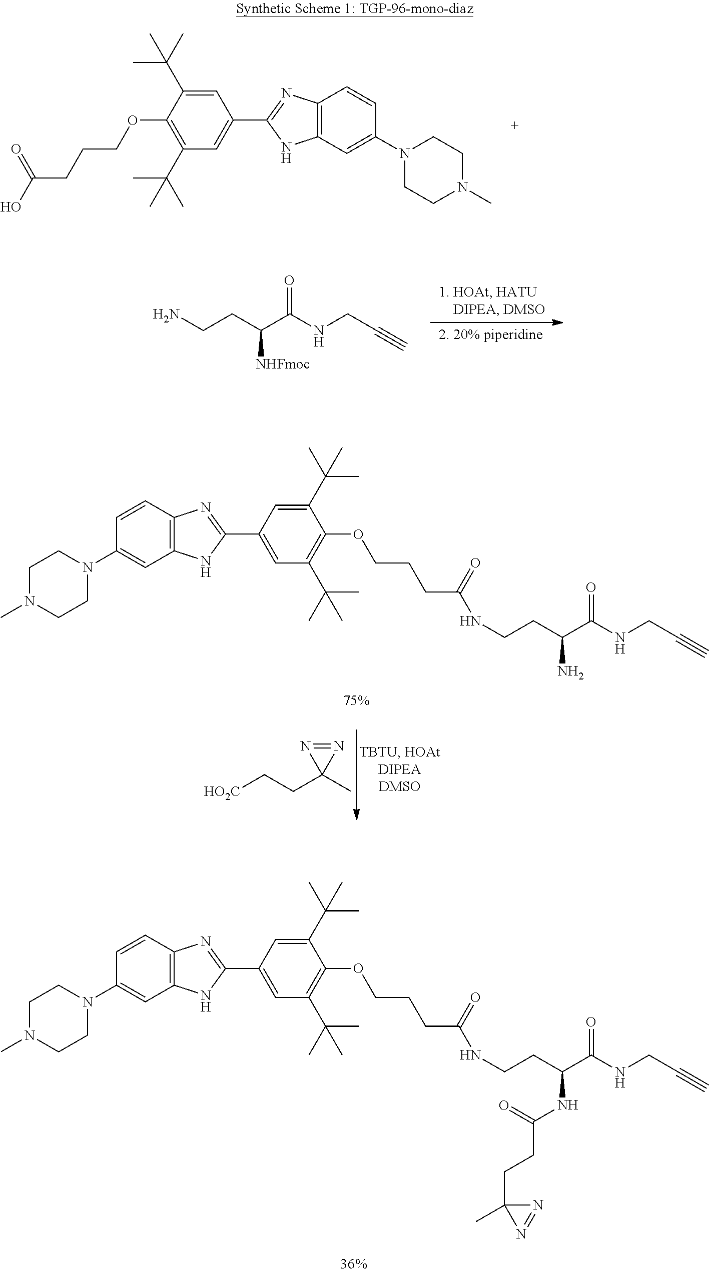

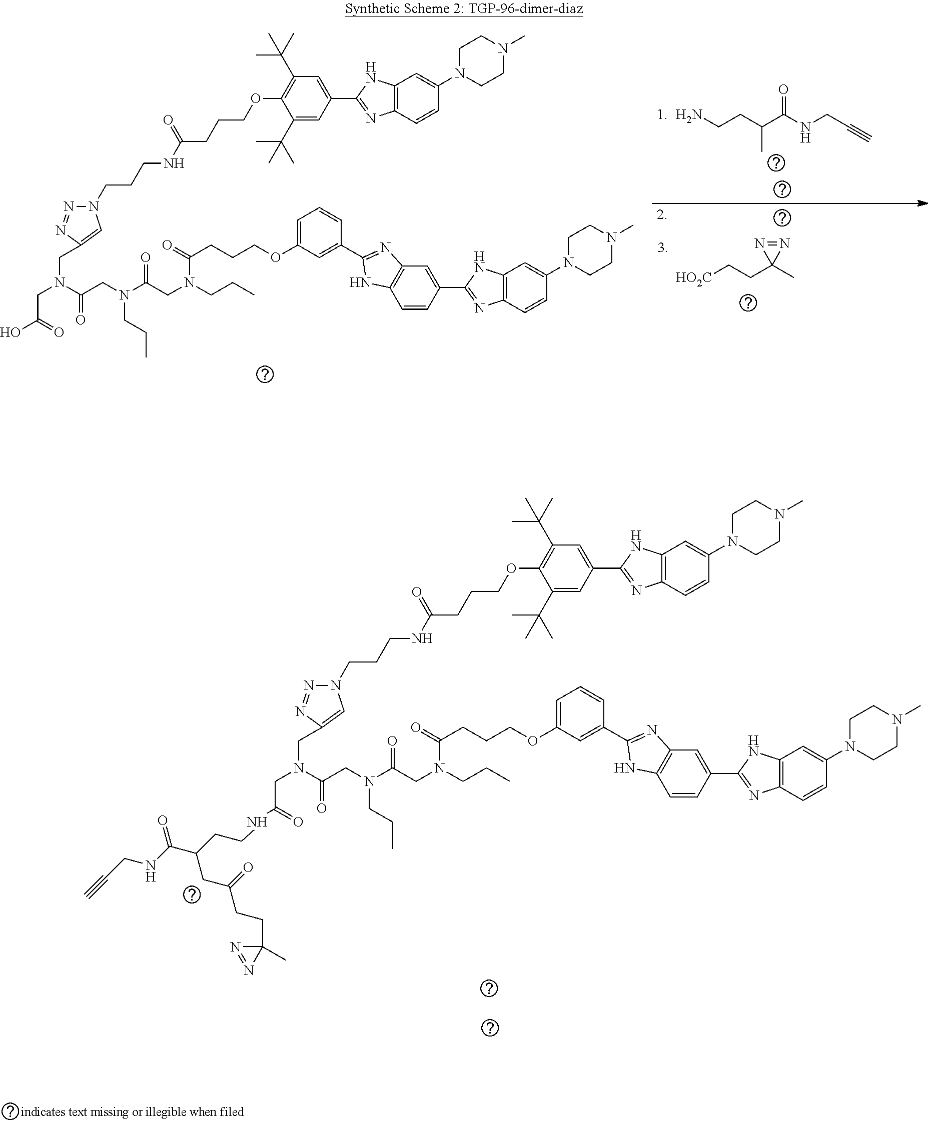

### Synthesis of Alkyne Reagent

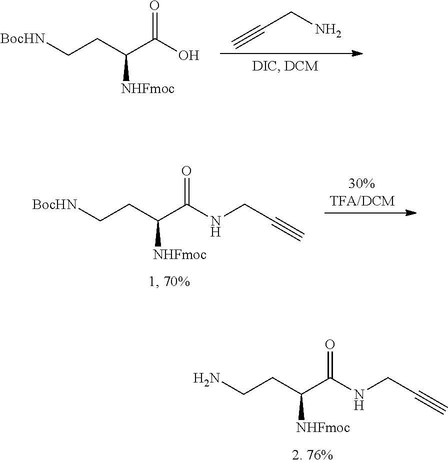

### Synthesis of Diazirine Carboxylic Acid

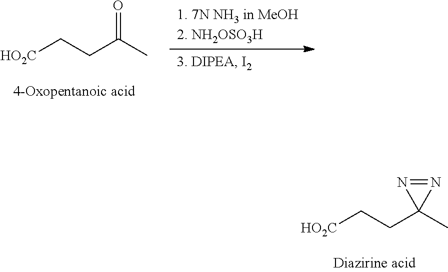

Synthesis of 1: Frnoc-Dab(Boc)-OH (CAS: 125238-99-5, 1 g, 2.27 mmol) was taken in a round bottom flask and was dissolved in 200 μL of dichloromethane (DCM). To this solution was added NAP-diisopropylcarbodiimide (DIC) (430 μL, 2.72 mmol). After stirring the reaction mixture at room temperature for 15 min, propargylamine (235 μL, 3.41 mmol) was added dropwise. The reaction was stirred at room temperature overnight, forming a white precipitate. After confirming the reaction went to completion by thin layer chromatography (TLC) and on reaction completion, the precipitate was filtered, washed with water, and dried. The white powder obtained was then purified by flash chromatography to obtain the desired product 1 (0.76 g; 70% yield).

Synthesis of 2: Compound 1 (700 mg) was placed in a round bottom flask with 50 μL of 30% trifluoracetic acid (TFA) in DCM. The solution was then stirred at room temperature for 30 min, Reaction progress was monitored by TLC and upon completion, the solvent was evaporated. The residue obtained was then dissolved in DCM and purified by flash chromatography to obtain desired product 2 (420 mg; 76% yield).

Synthesis of TGP-96-monomer-diaz TGP-96-COOH (7 mg,13.82 μmol), which was synthesized as previously described,¹ was dissolved in DMF (500 μL) containing HATU (10,51 mg, 27.63 μmol), HOAt (3.76 mg, 27.63 μmol), and DIPEA (8 μL,41.45 μmol). After stirring at room temperature for 15 min, compound 2 (7.82 mg, 20,72 μmol), dissolved in 50 μL DMF, was added to this reaction mixture, which was stirred at room temperature. Reaction progress was monitored via MALDI-TOF, and upon completion, the solvent was evaporated under vacuum. The residue obtained was purified using a Waters HPLC using a linear gradient of methanol in water (0 to 100%) over 60 min. The product's mass was confirmed via MALDI-TOF.

To the obtained product was added 20% piperidine in DMF (500 μL), and the reaction was stirred at room temperature for 30 min. Product formation was confirmed by MALDI-TOF analysis, and the solvent was evaporated under vacuum. The product obtained, 3 (6.6 mg, 10,25 μmol), was dissolved in DMF (400 μL) followed by addition of 100 μL of DMF containing TBTU (6.58 mg, 20.50 μmol), HOAt (2,79 mg, 20.50 μmol), DIPEA (3.8 μL, 20,50 μmol) and diazirine acid (1,97 mg, 15.38 μmol). The reaction was stirred at roorn temperature overnight, and the solvent was removed under vacuum. The residue was purified using HPLC as described for compound 2. The mass of desired product, TGP-96-diaz, was confirmed by MALDI-TOF.

Synthesis of TGP-96-dimer-diaz: TGP-96-dimer-COOH (8 mg,5.74 μmol), synthesized as previously described,¹ was dissolved in 250 μL of DMF. HATU (6.55 mg, 17.23 μmol), HOAt (2.35 mg, 17.23 μmol), and DIPEA (5.3 μL, 28.72 μmol) were then added, and the reaction mixture was stirred at room temperature for 15 min, After, compound 2 (3.25 mg, 8.62 μmol) dissolved in 50 μL DMF was added, and reaction was stirred again at room temperature. Reaction progress was monitored via MALDI-TOF MS, and, upon completion, the solvent was evaporated under vacuum. The residue obtained was purified by HPLC as described above. The product's mass was confirmed via MALDI-TOF MS.

The product was dissolved in 200 μL of 20% piperidine in DMF, and the reaction was stirred at room temperature for 30 min, Product formation was confirmed using MALDI-TOF MS, and the solvent was evaporated under vacuum. The resulting residue (3.74 mg, 2,42 μmol) was dissolved in DMF (100 μL) followed by addition of 100 μL of DMF containing TBTU (2.33 mg, 7.26 μmol), HOAt (987.5 pg, 7.26 μmol), DIPEA (1.34 pL, 7.26 μmol), and diazirine acid (929.6 pg, 7.26 μmol). The reaction was stirred at room temperature overnight, and product formation was confirmed by MALDI-TOF MS. The solvent was removed under vacuum, and the resulting residue was purified by HPLC as described above. The mass of desired product, TGP-96-dimer-diaz, was confirmed by MALDI-TOF MS.

Synthesis of control diaz: Compound 2 (360 mg, 953.8 μmol) was taken in a round bottom flask and DCM (50 μL) was added. Acetic anhydride (108 μL, 1.14 μmol) was added dropwise to the reaction followed by addition of triethylamine (146.6 pL, 1.05 μmol). The reaction mixture was stirred at room temperature overnight. Upon reaction completion, a white precipitate formed, which was filtered, washed with water, and dried, affording compound 4. To 4 was added 100 LL of 20% piperidine in DMF, and the reaction was stirred at room temperature for 30 min. After completion, the solvent was removed under reduced pressure, and the product was purified via flash chromatography. The product thus obtained (9.40 mg, 47.66 μmol) was dissolved in DMF (500 μL). To this was added 100 μL of DMF containing TBTU (2.33 mg, 7,26 μmol), HOAt (23,02 mg, 71.49 μmol), DIPEA (9.65 mg, 95.32 μmol) and diazirine acid (12.21 mg, 95.32 μmol). The reaction was stirred at room temperature overnight, with monitoring by thin layer chromatography (TLC). Upon reaction completion, the solvent was removed under vacuum, and the product was purified via flash chromatography.

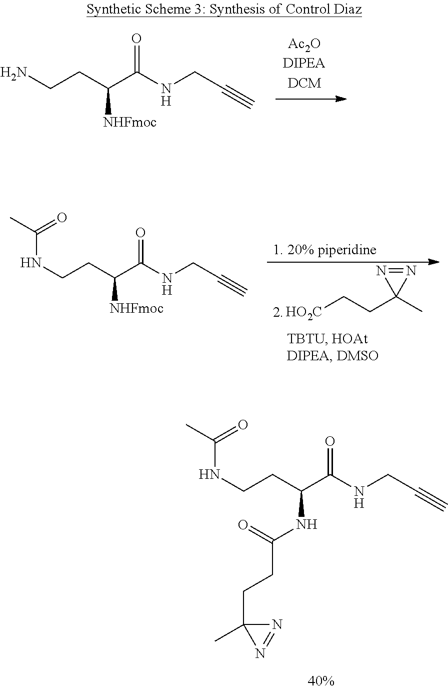

### In Vitro Chem-CLIP Studies

In vitro Chem-CLIP. In vitro transcribed pri-miR-96 was dephosphorylated with calf intestinal alkaline phosphatase [CIAP; New England Biolabs (NEB)], radiolabeled with [³²P-γ]ATP and T4 polynucleotide kinase (NEB), and purified by denaturing gel electrophoresis as previously described.² The RNA (5 μM final concentration) was folded in 10 μL of 20 mM Hepes buffer, pH 7,5, by heating to 60° C. for 5 min followed by slowly cooling to room temperature. The RNA solution was then treated with a Chem-CLIP probe of interest for 30 min at room temperature at varying concentrations to afford a dose response [for TGP-96-mono-diaz, 0.78 —50 pM; for TGP-96-dimer-diaz, 0.08-5 μM; and for Control-diaz (lacks RNA-binding modules; no reaction observed), 1-100 μM]. For competitive Chem-CLIP (C-Chem-CLIP) experiments, the RNA was incubated with a constant concentration of the diazirine derivative for 15 min at room temperature [for TGP-96-mono-diaz, 20 μM; for TGP-96-dimer-diaz, 2 μM]. The unreactive parent compound was then added at varying concentrations [for TGP-96-mono-diaz, 31-1000 μM; for TGP-96-dimer-diaz, 3-200 μM], and the samples were incubated for an additional incubation for 15 min at room temperature.

The samples were irradiated with UV light (365 nm) in a UVP Crosslinker (Analytik Jena) for 10 min. To each reaction was then added a “click mixture” [Biotin-PEG3-Azide (0,8 μL, 10 mM; Click Chemistry Tools), CuSO₄ (0.5 μL, 10 mM), Tris(3-hydroxypropyltriazolylmethyl)amine (THPTA; 0.1 μL, 50 mM) and sodium ascorbate (0.1 μL, 250 mM)]. The samples were incubated at 37° C. for 2 h followed by addition of 8 μL streptavidin beads (Dynabeads MyOne Streptavidin Cl beads; Thermo Scientific) to pull down crosslinked pri-miR-96. Un-reacted RNA, present in the supernatant, was removed by placing reaction tubes on a magnetic separation rack. The beads were washed twice with 1× Wash Buffer (10 mM Tris-HCl pH 7.0, 1 mM EDTA, 4 M NaCl, and 0.2% Tween). The radioactive signal associated with the beads and in the supernatant was measured by liquid scintillation counting.

Identification of compound binding sites via primer extension or sequencing. In vitro transcribed pri-miR-96 (100 μL of 5 μM) was folded in 20 mM Hepes, pH 7.5, as described above and treated with a diazirine Chem-CLIP probe [1 μM of TGP-96-dimer-diaz or 100 μM of TGP-96-mono-diaz] for 30 min at room temperature. The samples were then irradiated with UV light (365 nm) using a UVP Crosslinker for 10 min. Akin to the studies described above, the reaction was then supplemented with a click reaction mixture, in this case consisting of disulfide biotin azide (0.5 μL, 100 mM; Click Chemistry Tools), CuSO₄ (1 μL, 10 mM), THPTA (1 μL, 50 mM) and sodium ascorbate (1 μL, 250 mM). The reaction was incubated at 37° C. for 2 h, and the samples were ethanol precipitated. The RNA was reconstituted in 10 μL of Nanopure water and incubated with 10 μL of streptavidin beads (Dynabeads MyOne Streptavidin C1 beads; Thermo Scientific). After a 30 min incubation at room temperature, un-reacted RNA was removed by placing reaction tubes on a magnetic separation rack. The beads were washed twice with 1× Wash Buffer and then twice with Nanopure water.

The cross-linked RNA associated with the beads was eluted by reducing the disulfide bond present in the biotin used in the click reaction mixture. In brief, the beads were incubated with 50 μL of a prepared mixture containing 1:1 200 mM tris(2-carboxyethyl)phosphine (TCEP) and 600 mM K₂CO₃ for 30 min at 37° C. The thiols were then capped with iodoacetamide (50 μL, 400 mM) for 30 min at room temperature (protected from light), The beads were removed using a magnetic rack, and the eluted RNA in the supernatant was transferred to a new tube and ethanol precipitated.

Compound binding sites can be identified by reverse transcription (RT). That is, RT is unable to proceed through nucleotides where the compound has been cross-linked. Two different methods were employed to identify these “RT Stops”: primer extension using a radioactively labeled, gene-specific RT primer and ligation-mediated PCR and sequencing. For analysis by primer extension, RT was carried out using

SEQ ID: 1
5′-³²P-CAGACGTGCTCTTCCGATCTCGCAGCTGCGGGTCCT,,

and Superscript III (SSIII) per manufacturer's protocol (500 ng of RNA in a total reaction volume of 20 μL). The reaction was incubated at 55° C. for 50 min, followed by inactivation at 85° C. for 5 min. The RT products were separated on a denaturing 15% polyacrylamide gel to identify “RT stops” and hence compound binding sites; dideoxy sequencing samples were run in parallel. The gel was imaged using a Molecular Dynamics Typhoon 9000 phosphorimager and quantified with BioRad's QuantityOne software.

For ligation-mediated PCR, or Chem-CLIP-Map-Seq, gene-specific RT was completed as described above except the primer was not radioactively labeleld. After the RT step, the RNA was digested with 0.5 μL RNase H and 0.5 μL RNase A for 30 min at 37° C. The RT reaction was then cleaned up using Agencourt RNAClean XP beads per manufacturer's protocol for small RNAs. The purified RT reaction was ligated to a ssDNA (5′Phos-NNNAGATCGGAAGAGCGTCGTGTAG-3Cspacer), SEQ ID:2, using T4 RNA ligase I (NEB). Briefly, 2 μL 10×T4 RNA ligase buffer, 1 μL 1 mM ATP, 10 μL 50% PEG 8000, 5 μL cDNA, 1 μL of 20 μM ssDNA adaptor, and 1 μL of T4 RNA ligase were incubated overnight at RT.³ This reaction mixture was cleaned up using RNAClean XP as described above for RT reactions. The ligated cDNA was amplified using the following primers and Phusion polyrnerase (NEB): 5′-CTACACGACGCTCTTCCGATCT-3′ and 5′-CAGACGTGCTCTTCCGAT-3′, SEQ ID:3. The PCR products and primers were separated on a denaturing 12% polyacrylamide gel containing SYBR green. The desired band was excised, and the DNA was extracted was isolated by tumbling the gel overnight in 0.3 M sodium chloride solution overnight followed by ethanol precipitation. The isolated cDNA was then cloned into NEB's pminiT 2.0 vector per manufacturer's protocol and sequenced by Eton Biosciences.

### Cellular Chem-CLIP Studies.

An appropriate cell line (in 100 mm dishes) was treated with a diazirine-containing Chem-CLIP probe at varying concentrations for an appropriate amount of time (typically 30 min-8 h), After the treatment period, the growth medium was removed, and the cells were washed with ice-cold lx PBS. Ice-cold 1× PBS was then added to the culture flask (20 mL), and the cells were irradiated for 20 min with 365 nm light using a UVP Crosslinker. After scraping the cells into ice-cold 1× PBS and pelleting them by centrifugation, total RNA was harvested using a Qiagen RNeasy Kit per the manufacturer's recommended protocol.

A 90 μg sample of total RNA was dissolved in 150 μL of RNase/iDNase free water. To this was added Hepes buffer, pH 7,1, to a final concentration of 25 mM. Using a GenElute filtration column (Sigma Aldrich), disulfide azide agarose beads (1 mL, Click Chemistry Tools) were washed three times with 25 mM Hepes, pH 7.1, and then resuspended in 1.5 mL of 25 mM Hepes, pH. 7.1. A 150 μL aliquot of the slurry was added to the total RNA, followed by 30 μL of 1:1:1; Ascorbate:CuSO₄:THPTA. The slurry was shaken at 37° C. for 2 h and transferred to a GenElute column to remove the reaction solution. The beads in the column were washed with 1× Wash Buffer three times and water twice. Then, 150 μL of a 1:1 mixture of TCEP, reduced with K₂CO₃ was added, and the sample was incubated at 37° C. for 30 min with vigorous shaking. This was followed by addition of 1 volume of iodoacetamide, and the samples were incubated for an addition 30 min at room temperature. The beads were then filtered using a GenElute filtration column by centrifugation and washed two times with Nanopure water. The eluent was concentrated en vacuo to ˜270 μL followed by addition of 270 μL of 100% Isopropanol and 270 μL of RNAClean XP beads, mixing by pipetting up and down 10 times. The beads were captured and washed twice with 85% ethanol and air dried. The RNA was eluted by adding 50 μL of DNase/RNase free water (Zymo). This elution step was then repeated after the beads were incubated for 2 min with an additional 50 μL of DNase/RNase free water, The eluted RNA was pooled and cleaned up using RNAClean XP beads per the manufacturers protocol.

Total RNA was quantified using Nanodrop UV spectrophotometer (ThermoFisher), A NEBNext Ultra II Directional RNA kit (Catalog #: E7760, NEB) was used for library preparation according to the manufacturer's instructions. RNA samples were then chemically fragmented in a divalent cation buffer with heating at 94° C. for 15 min. Random hexamer priming and reverse transcription were used to convert the fragmented RNA to the first strand of cDNA. The second strand was synthesized by removing the RNA template and incorporating dUTP in place of dTTP, which was then end repaired and adenylated at the 3′ end. A corresponding T nucleotide on the hairpin loop adaptor was used to ligate to the double-stranded cDNA. Uracil-specific excision reagent (USER) enzyme was then used to remove the dUTP in the loop, as well as other incorporated U's in the second strand. Degradation of the second strand preserves the directional sequencing of the original RNA, The adaptor ligated DNA was PCR amplified with Illumina barcoding primers to generate the final libraries, where only library fragments with both 5′ and 3′ adaptors would be enriched in the final PCR step, After validation on a Bioanalyzer DNA chip, the final libraries were normalized to 2 nM, pooled equally and sequenced on a Nextseq 500 v2.5 flow cell at 1.8 pM final concentration using 2×40 bp paired-end chemistry.

What is claimed is:

1. A method of determining the binding site on an RNA molecule to which a small molecule interacting with RNA (SMIRNA) associates, comprising
forming a derivative of the SMIRNA comprising the small molecule covalently coupled with a moiety comprising a diazirine group and a moiety comprising an alkyne group; then,
contacting the RNA with the SMIRNA derivative and exposing to UV light to activate the diazirene and bring about covalent coupling of the SMIRNA derivative to the RNA forming a SMIRNA-RNA covalent complex; then,
contacting the SMIRNA-RNA covalent complex with an azide-bearing group further comprising a pull-down group comprising a biotin moiety linked to the azide by a linker group comprising a disulfide bond, or alternatively with a bead bearing an azide group bonded thereto by a linker comprising a disulfide bond, under conditions comprising the presence of copper catalyst to bring about a CuAAC reaction; then,
contacting the SMIRNA-RNA covalently bonded to the biotin moiety and a complementary pull-down entity comprising streptavidin immobilized on a bead to isolate the SMIRNA-RNA covalent complex; then,
cleaving the SMIRNA-RNA covalent complex from the bead by reduction of the disulfide bond in the respective linker, to free the complex from the bead; then,
subjecting the RNA thus freed to reverse transcription and sequencing of the DNA from the reverse transcription process, to indicate the SMIRNA binding site on the RNA through the presence of a transcription stop.

2. The method of claim 1 wherein the SMIRNA is SMIRNA-1 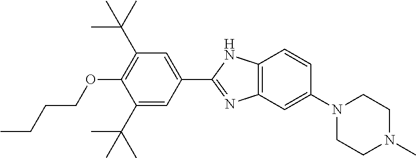 [SMILES: CCCCOc1c(C(C)(C)C)cc(-c2nc3cc(N4CCN(C)CC4)ccc3[nH]2)cc1C(C)(C)C]
or SMIRNA-2

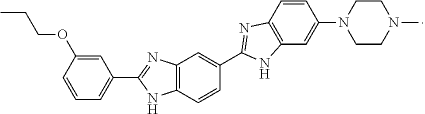 [SMILES: CCCOc1cccc(-c2nc3cc(-c4nc5ccc(N6CCN(C)CC6)cc5[nH]4)ccc3[nH]2)c1]

3. The method of claim 1 wherein the SMIRNA derivative is TGP-9 ono-diaz

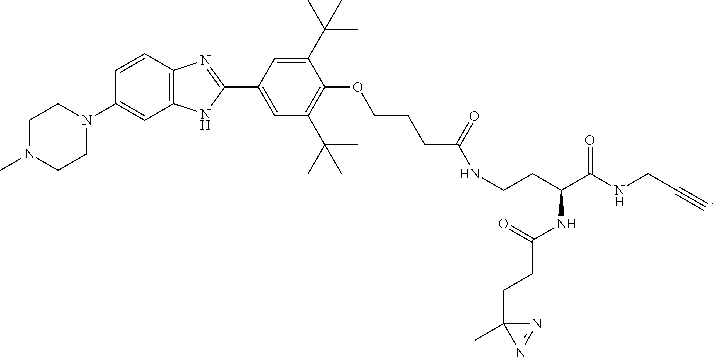 [SMILES: C#CCNC(=O)[C@H](CCNC(=O)CCCOc1c(C(C)(C)C)cc(-c2nc3ccc(N4CCN(C)CC4)cc3[nH]2)cc1C(C)(C)C)NC(=O)CCC1(C)N=N1]

4. The method of claim 1 wherein the SMIRNA derivative is TGP-96-dirner-diaz

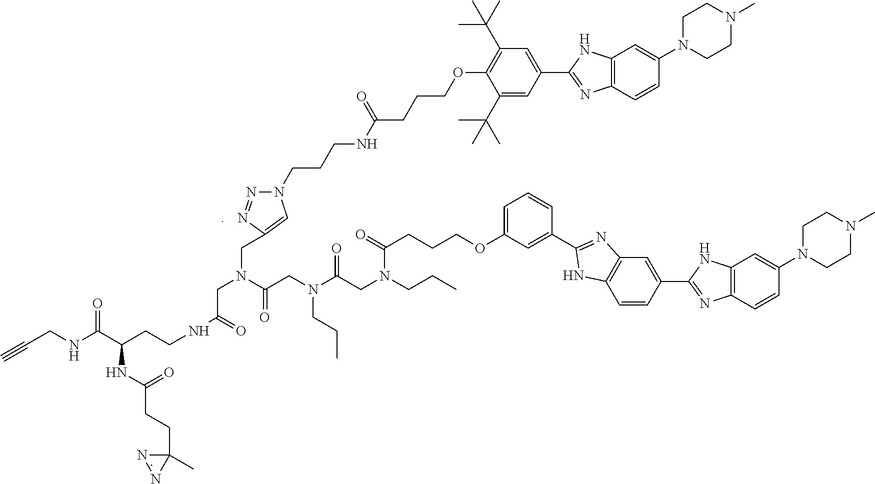 [SMILES: C#CCNC(=O)[C@@H](CCNC(=O)CN(Cc1cn(CCCNC(=O)CCCOc2c(C(C)(C)C)cc(-c3nc4ccc(N5CCN(C)CC5)cc4[nH]3)cc2C(C)(C)C)nn1)C(=O)CN(CCC)C(=O)CN(CCC)C(=O)CCCOc1cccc(-c2nc3cc(-c4nc5ccc(N6CCN(C)CC6)cc5[nH]4)ccc3[nH]2)c1)NC(=O)CCC1(C)N=N1]

5. The method of claim 1 wherein the azide group of the CuAAC reaction is linked to a biotin group.

6. The method of claim 1 wherein the azide group of the CuAAC reaction is linked directly to a bead.

7. The method of claim 1, wherein the identifying comprises
(1) reverse transcribing the RNA from the purified complex with a primer to create cDNA;
(2) amplifying the cDNA with a primer set suitable for RT-qPCR or high-throughput sequencings (RNA-seq); and
(3) analyzing the cDNA to identify reverse transcriptase sequence stops to thereby determine the sequence of the binding site of the RNA-binding module on the RNA.

8. The method of claim 1, wherein the RNA cross-linking module comprises a diazirine group.

9. The method of claim 1, wherein the purification module comprises a biotin group.

10. The method of claim 1, wherein the RNA comprises an oncogenic non-coding RNA precursor, and the contacting of step (a) comprises contacting a cell expressing the non-coding RNA precursor and the compound.

11. The method of claim 1, wherein the oncogenic non-coding RNA precursor comprises oncogenic primary microRNA-96 (pri-miR-96),

12. The method of claim 1, wherein the RNA comprises a transcriptome.

13. The method of claim 1, wherein the transcriptome is viral.

14. The method of claim 1, wherein the transcriptome is mammalian.

15. The method of claim 1, wherein the transcriptome is bacterial.

16. The method of claim 1, wherein the RNA comprises one or more of synthetic, semi-synthetic, or natural RNA.

17. The method of claim 1, wherein the RNA comprises the genome of an RNA virus.

18. The method of claim 1 carried out in vitro.

19. The method of claim 1 carried out in living cells.

20. The method of claim 1 carried out in a preclinical animal model.

21. The method of claim 1, wherein the living cells are virally- or bacterially-infected cells.

22. The method of claim 1, wherein the method is performed in a parallel array for a plurality of RNA sequences and candidate RNA-binding small modules.
